(19) United States
(12) Patent Application Publication
(10) Pub. No.: US 2026/0102092 A1
(43) Pub. Date: Apr. 16, 2026

(54) COGNITIVE IMPAIRMENT DIAGNOSIS METHOD BASED ON EYE TRACKING OF WEBCAM

(71) Applicant: Wenzhou Baoying Technology Co., Ltd., Wenzhou (CN)

(72) Inventor: Chenkai ZHANG, Wenzhou (CN)

(73) Assignee: Wenzhou Baoying Technology Co., Ltd., Wenzhou (CN)

(21) Appl. No.: 19/034,644

(22) Filed: Jan. 23, 2025

(30) Foreign Application Priority Data
Oct. 11, 2024 (CN) 202411419800.7

(51) Int. Cl. A61B 5/16; A61B 5/00; G16H 10/60; G16H 50/20

(52) U.S. Cl. CPC A61B 5/165; A61B 5/163; A61B 5/4088; G16H 10/60; G16H 50/20

(57) ABSTRACT

A cognitive impairment diagnosis method based on eye tracking of a webcam includes: S1, acquiring user data, and performing a numbering processing on a user to generate a user identity document (ID); S2, performing an eye movement test on the user, collecting an eye movement photo by using a webcam, and processing the eye movement photo to obtain sample data; S3, constructing an eye movement model, processing sample data through the eye movement model to obtain a gaze trajectory, wherein the gaze trajectory comprises a plurality of gaze site coordinates; S4, processing the plurality of gaze site coordinates to obtain sample coordinates, constructing a transformer model, inputting the sample coordinates to evaluate the user and outputting an early screening diagnosis report of cognitive impairment. The method can reduce the technical obstacles of early Alzheimer's disease detection, and is suitable for routine screening of cognitive impairment in various scenarios.

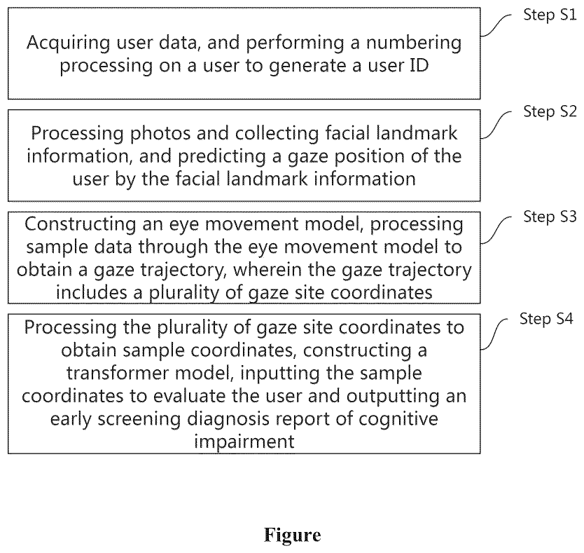

### CROSS REFERENCE TO THE RELATED APPLICATIONS

This application is based upon and claims priority to Chinese patent application No. 202411419800.7, filed on Oct. 11, 2024, the entire contents of which are incorporated herein by reference.

### TECHNICAL FIELD

The present invention relates to the field of cognitive impairment detection, in particular to a cognitive impairment diagnosis method based on eye tracking of a webcam.

### BACKGROUND

The mainstream methods for diagnosing cognitive impairment include neuropsychological tests and biomarker analysis. Although these methods are widely used, they are invasive, and costly and operational complexity, especially in a resource-limited environment. The present invention significantly improves the convenience and popularity of diagnostic technology by using ordinary webcams and algorithms. Currently, the eye tracking technology used in the diagnosis of cognitive impairment usually includes two main directions: one is to test the response delay by performing tasks such as reverse saccades, and another one is to analyze the eye movement heat map after viewing a specific stimulus. These tasks usually require participants to carry out pre-learning, which is particularly inconvenient in areas with low education levels or language barriers.

In addition, although the heat map analysis is simple to operate, the potential of these data is not fully utilized due to the time series information in the eye movement data not being fully explored. These two methods also rely on expensive eye tracking equipment, which limits the popularity of the equipment in economically underdeveloped areas.

Therefore, a cognitive impairment diagnosis method based on eye tracking of a webcam is urgently needed to solve the above problems.

### SUMMARY

In order to solve the above problems, the present invention provides a cognitive impairment diagnosis method based on eye tracking of a webcam, whether participants have potential dementia is screened by using the basic nature of gaze data.

A cognitive impairment diagnosis method based on eye tracking of a webcam provided by the present invention includes the following steps:
S1, acquiring user data, and performing a numbering processing on a user to generate a user identity document (ID);
S2, processing photos and collecting facial landmark information, and predicting a gaze position of the user by the facial landmark information;
S3, constructing an eye movement model, processing sample data through the eye movement model to obtain a gaze trajectory, wherein the gaze trajectory includes a plurality of gaze site coordinates;
S4, processing the plurality of gaze site coordinates to obtain sample coordinates, constructing a transformer model, inputting the sample coordinates to evaluate the user and outputting an early screening diagnosis report of cognitive impairment.

Preferably, a content of eye movement test for users in S2 specifically includes: a matrix part and an art part;
the matrix part includes a randomly generated matrix of 5×5 dots, and a subject is required to calculate a number of dots;
the art part includes a face image, the face image includes several faces, the subject is required to count a number of faces in the image.

Preferably, a specific content of constructing the eye movement model in S3 is:
collecting a plurality of facial pictures, and performing a feature analysis on the plurality of facial pictures to obtain sample facial features, wherein the sample facial features include a key coordinate of a head and a position of a pupil in an eye;
calculating a head rotation vector by a solvepnp function;
establishing a corresponding relationship for every two of the key coordinates of the head, the position of the pupil in the eye and the head rotation vector;
training the eye movement model according to the corresponding relationship;
taking the sample data into the eye movement model, extracting the facial features, and obtaining the gaze trajectory.

Preferably, a specific content of processing the plurality of gaze site coordinates to obtain sample coordinates, constructing a transformer model, inputting the sample coordinates to evaluate the user and outputting an early screening diagnosis report of cognitive impairment is:
evaluating a cognitive level of the user by using a mini-mental state examination (MMSE) method, and using the score as a training set label to train the Transformer model;
dividing the sample coordinates into five parts by the transformer model, using four parts as training data and one part as verification data, and performing a 5-fold cross-validation with Area Under Curve (AUC) as the standard.

Preferably, the transformer model inputs a gaze point and a displayed image with dimensions of x and y coordinates;

the transformer model has a model dimension of 64, a number of layers of 3, a head nhead of 8, a feedforward dimension of 256, a loss function of BCELoss, and a training period of 100.

Preferably, a specific content of evaluating the user by the mini-mental state examination method is:
presetting a target score limit value, and if the score value is greater than or equal to the target score limit value, it is judged that the user has cognitive impairment;
if the score value is less than the target score limit value, it is judged that the user has no cognitive impairment.

In summary, a cognitive impairment diagnosis method based on eye tracking of a webcam provided by the present invention, compared with conventional technology that uses an ordinary webcam instead of professional eye tracking equipment, greatly reduces the cost, and the use of simple equipment and algorithm makes the use of this technology is easier to popularize and applied to various scenarios, and provides a kind of operation suggestion, non-invasive cognitive impairment diagnosis method. Non-invasive and non-contact increase the willingness of a user to see a doctor, and reduces the low cost of treatment for the user without professional equipment.

Further detailed descriptions of a technical scheme of the present invention can be found in the accompanying drawings and embodiments.

### BRIEF DESCRIPTION OF THE DRAWINGS

FIGURE is a flowchart of a cognitive impairment diagnosis method based on eye tracking of a webcam according to the present invention.

### DETAILED DESCRIPTION OF THE EMBODIMENTS

Hereinafter, the technical solution of the present invention will be further described with reference to the accompanying drawings and embodiments. It should be noted that the relative arrangements of components and steps, numerical expressions, and numerical values set forth in these examples do not limit the scope of the present invention unless specifically stated otherwise.

The following description of at least one illustrative embodiment is actually only illustrative and in no way a restriction on the invention and its application or use.

The technology, systems and equipment known to ordinary technicians in related fields may not be discussed in detail, but in appropriate cases, technology, systems and equipment should be considered as part of the specification.

In all the examples shown and discussed here, any specific value should be interpreted as merely illustrative, not as a restriction. Therefore, other examples of exemplary embodiments can have different values.

Unless otherwise defined, technical or scientific terms used in the present invention are to be given their ordinary meaning as understood by those of ordinary skill in the art to which the present invention belongs.

As shown in the FIGURE, the present invention provides a cognitive impairment diagnosis method based on eye tracking of a webcam, as follows, S1, user data is acquired, and a numbering processing is performed on a user to generate a user identity document (ID);
S2, photos are processed and facial landmark information is collected, and a gaze position of the user is predicted by the facial landmark information;
preferably, a content of eye movement test for users in S2 specifically includes: a matrix part and an art part;
the matrix part includes a randomly generated matrix of 5×5 dots, and a subject is required to calculate a number of dots;
the art part includes a face image, the face image includes several faces, the subject is required to count a number of faces in the image.

S3, an eye movement model is constructed, sample data is processed through the eye movement model to obtain a gaze trajectory, wherein the gaze trajectory includes a plurality of gaze site coordinates;
preferably, a specific content of constructing the eye movement model in S3 is:
a plurality of facial pictures is collected, and a feature analysis is performed on the plurality of facial pictures to obtain sample facial features, wherein the sample facial features include a key coordinate of a head and a position of a pupil in an eye;
a head rotation vector is calculated by a solvepnp function;
a corresponding relationship is established for every two of the key coordinates of the head, the position of the pupil in the eye and the head rotation vector;
the eye movement model is trained according to the corresponding relationship;
the sample data is taken into the eye movement model, the facial features are extracted, and the gaze trajectory is obtained.

Preferably, a content of taking the sample data into the eye movement model, extracting the facial features, and obtaining the gaze trajectory is:
the collected eye movement photos are cleaned, corrected and synchronized;
key facial feature coordinates are extracted by using dlib's 68-point facial landmark detection model after capturing facial images through the webcam;
3D coordinate points of a key area of the face are constructed, and the rotation vector from the 2D image points to the 3D model is calculated by using the EPnP projection in the solvePnP algorithm of OpenCV;
the orientation of the head in the three-dimensional space is obtained according to the rotation vector;
the gaze coordinates are obtained by extracting a relative position of the pupil in the eye through the GazeTracking library.

S4, the plurality of gaze site coordinates are processed to obtain sample coordinates, a transformer model is constructed, the sample coordinates are input to evaluate the user and a screening diagnosis report of cognitive impairment is output.

Preferably, a specific content of processing the plurality of gaze site coordinates to obtain sample coordinates, constructing a transformer model, inputting the sample coordinates to evaluate the user and outputting a screening diagnosis report of cognitive impairment is:
a cognitive level of the user is evaluated by using the MMSE method, and the score is used as a training set label to train the Transformer model;
the sample coordinates are divided into five parts by the transformer model, four parts are used as training data and one part as verification data, and a 5-fold cross-validation is performed with AUC as the standard.

Preferably, the transformer model inputs a gaze point and a displayed image with dimensions of x and y coordinates;
the transformer model has a model dimension of 64, a number of layers of 3, a head nhead of 8, a feedforward dimension of 256, a loss function of BCELoss, and a training period of 100.

Preferably, a specific content of evaluating the user by the mini-mental state examination method is:
a target score limit value is preset, and if the score value is greater than or equal to the target score limit value, it is judged that the user has cognitive impairment;
if the score value is less than the target score limit value, it is judged that the user has no cognitive impairment.

The information table of the subjects obtained from the Mini-Mental State Examination (MMSE) questionnaire of the mental state examination score is as follows:

TABLE 1

| | | | |
|---|---|---|---|
| | Proportion of | | |
| All participants | participant | MCI | HC |
| of the attribute | categories | group | group |

Age

| | | | |
|---|---|---|---|
| 45-55 | 12.7% | 3.3% | 7.4% |
| 55-64 | 26.7% | 16.7% | 40.7% |
| 65-74 | 37.5% | 53.3% | 40.7% |
| 75+ | 23.2% | 26.7% | 11.1% |

Gender

| | | | |
|---|---|---|---|
| Male | 43.4% | 40.0% | 55.6% |
| Female | 56.6% | 60.0% | 44.4% |

Education

| | | | |
|---|---|---|---|
| Illiterate | 35.6% | 56.7% | 22.2% |
| Primary school | 45.2% | 33.3% | 37.0% |
| Junior high school | 15.8% | 6.7% | 29.6% |
| High school | 3.4% | 3.3% | 11.1% |

The healthy control group (HC) (=27) and mild cognitive impairment (MCI) patients (=30) were determined based on the participants' MMSE assessment scores. Gender distribution showed that women's cognitive function was usually poor, but the difference was not statistically significant. Participants in the HC group tended to be younger than those in the MCI group, and the difference was statistically significant. The mean MMSE score was 27.3 in HC patients and 19.3 in MCI patients. The education level of the HC group and the MCI group was also different, and there was a statistical difference between the two groups.

In this present invention, a using model is introduced to process eye tracking data to detect MCI. The model showed good ability in classifying participants with cognitive impairment and normal controls, and achieved a significant area under curve (AUC).

The performance of the model was evaluated based on precision, accuracy, recall, and F1 score, the ROC-AUC.F1score was used to evaluate the balance between precision and accuracy of the model.

The expression of the F1 score is:

$${F}_{1} = 2 \cdot \frac{precision \cdot recall}{precision + recall} ;$$

the comparative analysis with previous studies showed that the model in this present invention has achieved significant improvement in performance. The AUC range of previous studies is 0.61-0.85, which is lower than 0.92 of the present invention.

Even compared with recent research using advanced deep learning methods, the model of the present invention still achieves better performance. As shown in Table 2, the AUC of the study using VTnet is 0.75, while the AUC of the present invention is 0.92. The f1 score of the research using MC-CNN is 0.81, while the f1 score of the present invention is 0.97.

TABLE 2

| | Laser scanning | Random | | | Model used in the |
|---|---|---|---|---|---|
| Indicators | tracking method | forest | XGBoost | Transformer | present invention |
| Precision | 0.65 | 0.7030 | 0.6485 | 0.7841 | 0.917 |
| Accuracy rate | 0.5724 | 0.7000 | 0.6333 | 0.8000 | 0.940 |
| Recall rate | 0.96 | 0.7248 | 0.6248 | 0.7595 | 1.000 |
| F1 | 0.7119 | 0.6900 | 0.6134 | 0.7542 | 0.965 |
| AUC | 0.7081 | 0.7168 | 0.6593 | 0.8644 | 0.924 |

In the present invention, the use of webcam-based eye tracking can also serve as a proof-of-concept for a more readily available MCI screening tool. Although data collected from webcams provides lower fidelity compared to specialized eye tracking hardware, they significantly reduce the cost and complexity of setup. The present invention proves that even if there is a low-quality input, the model of the present invention can achieve a high level of diagnostic accuracy, making it suitable for routine screening of cognitive impairment in various scenarios such as home, medical, and healthcare institutions.

Finally, it should be noted that the above examples are merely used for describing the technical solutions of the present invention, rather than limiting the same. Although the present invention has been described in detail with reference to the preferred examples, those of ordinary skill in the art should understand that the technical solutions of the present invention may still be modified or equivalently replaced. However, these modifications or substitutions should not make the modified technical solutions deviate from the spirit and scope of the technical solutions of the present invention.

1. A cognitive impairment diagnosis method based on eye tracking of a webcam, comprising the following steps:
S1, acquiring user data, and performing a numbering processing on a user to generate a user identity document (ID);
S2, processing photos and collecting facial landmark information, and predicting a gaze position of the user by the facial landmark information;
S3, constructing an eye movement model, processing the sample data through the eye movement model to obtain a gaze trajectory, wherein the gaze trajectory comprises a plurality of gaze site coordinates;
S4, processing the plurality of gaze site coordinates to obtain sample coordinates, constructing a transformer model, inputting the sample coordinates to evaluate the user and outputting an early screening diagnosis report of cognitive impairment.

2. The cognitive impairment diagnosis method based on eye tracking of the webcam according to claim 1, wherein a content of the eye movement test for users in S2 specifically comprises: a matrix part and an art part;
the matrix part comprises a randomly generated matrix of 5×5 dots, and a subject is required to calculate a number of dots;
the art part comprises a face image, the face image comprises several faces, the subject is required to count a number of faces in the image.

3. The cognitive impairment diagnosis method based on eye tracking of the webcam according to claim 2, wherein a specific content of constructing the eye movement model in S3 is:
collecting a plurality of facial pictures, and performing a feature analysis on the plurality of facial pictures to obtain sample facial features, wherein the sample facial features comprise a key coordinate of a head and a position of a pupil in an eye;
calculating a head rotation vector by a solvepnp function;
establishing a corresponding relationship for every two of the key coordinates of the head, the position of the pupil in the eye and the head rotation vector;
training the eye movement model according to the corresponding relationship;
taking the sample data into the eye movement model, extracting the facial features, and obtaining the gaze trajectory.

4. The cognitive impairment diagnosis method based on eye tracking of the webcam according to claim 3, wherein a specific content of processing the plurality of gaze site coordinates to obtain the sample coordinates, constructing the transformer model, inputting the sample coordinates to evaluate the user and outputting the early screening diagnosis report of cognitive impairment is:
evaluating a cognitive level of the user by using a mini-mental state examination (MMSE) method, and using the score as a training set label to train the Transformer model;
dividing the sample coordinates into five parts by the transformer model, using four parts as training data and one part as verification data, and performing a 5-fold cross-validation with Area Under Curve (AUC) as the standard.

5. The cognitive impairment diagnosis method based on eye tracking of the webcam according to claim 4, wherein the transformer model inputs a gaze point and a displayed image with dimensions of x and y coordinates;
the transformer model has a model dimension of 64, a number of layers of 3, a head nhead of 8, a feedforward dimension of 256, a loss function of BCELoss, and a training period of 100.

6. The cognitive impairment diagnosis method based on eye tracking of the webcam according to claim 5, wherein a specific content of evaluating the user by the mini-mental state examination method is:
presetting a target score limit value, and if the score value is greater than or equal to the target score limit value, it is judged that the user has cognitive impairment;
if the score value is less than the target score limit value, it is judged that the user has no cognitive impairment.
